(19) United States
(12) Patent Application Publication
(10) Pub. No.: US 2003/0003515 A1
(43) Pub. Date: Jan. 2, 2003

(54) Monocloral antibody-based diagnostic assay for gamma fibrinogen

(76) Inventors: David H. Farrell, University Park, PA (US); Hamid A. Al-Mondhiry, University Park, PA (US)

Correspondence Address: MCKEE, VOORHEES & SEASE, P.L.C., ATTN: PENNSYLVANIA STATE UNIVERSITY, 801 GRAND AVENUE, SUITE 3200, DES MOINES, IA, 50309-2721, US

(21) Appl. No.: 10/006,403

(22) Filed: Dec. 5, 2001

Related U.S. Application Data
(60) Provisional application No. 60/251,291, filed on Dec. 5, 2000.

(51) Int. Cl. G01N 33/53; G01N 33/542; G01N 33/537; G01N 33/543

(52) U.S. Cl. 435/7.9; 435/7.92

(57) ABSTRACT

The present invention relates to the novel finding that that elevated plasma levels of γA/γ′ are an independent risk factor for coronary artery disease. The invention therefore includes a method and diagnostic kit for the detection of γA/γ′ levels in patients. The kit preferably contains an antibody molecule or fragment thereof that is capable of specifically immunoreacting with a binding site in γA/γ′. Most preferably, the monoclonal antibody is capable of immunoreacting with the twenty carboxyl terminal amino acids of the γ′ chain of γA/γ′.

### PRIORITY CLAIM

This application claims priority to U.S. Provisional Application Serial No. 60/251,291 filed Dec. 5, 2000.

### FIELD OF THE INVENTION

This invention relates to diagnostic assays for the detection of γA/γ′ fibrinogen for the prevention of coronary artery disease. This invention further relates to monoclonal antibodies specific to gamma fibrinogen.

### BACKGROUND OF THE INVENTION

Coronary artery disease, also called coronary heart disease or heart disease, is the leading cause of death for both men and women in the United States. According to the American Heart Association, in 1995 one in every 4.8 deaths in the United States was caused by coronary artery disease. Fourteen million Americans have active symptoms of coronary artery disease (heart attack or chest pains). Many million more have silent coronary disease, the first indication of which can be sudden death.

Coronary artery disease is a narrowing or blockage of the arteries and blood vessels that provide oxygen and nutrients to the heart. It is caused by atherosclerosis, an accumulation of cholesterol and other fatty substances on the inner linings of arteries. These substances attract fatty tissue, blood components, and calcium and harden into artery-clogging plaques. The resulting blockage restricts blood flow to the heart. When the blood flow is completely cut off, the result is a heart attack.

The incidence of heart disease has been correlated with several risk factors, including heredity, smoking, high cholesterol, high blood pressure, and sedentary life style. Another identified risk factor is high plasma fibrinogen levels.^{1,2} Fibrinogen is a large plasma protein involved in blood clotting. During the clotting process, fibrinogen is converted into a clot by the action of a proteolytic enzyme, thrombin, in the presence of several other accessory factors.

The mechanism by which elevated fibrinogen levels contribute to heart disease is unclear. Several explanations have been suggested, including increased plasma viscosity, platelet hyperaggregability, and increased atherosclerosis. It has also been suggested that increased fibrinogen is simply a marker of chronic inflammation, since fibrinogen is an acute phase protein.³ Establishing a mechanism, however, is complicated by the fact that plasma fibrinogen is a heterogeneous mixture of isoforms with differing polypeptide compositions and bioactivities.

The most common form of fibrinogen consists of three polypeptide chains, α (alpha), β (beta), and γ (gamma), arranged as a dimer with the stoichiometry (α, β, γ)₂. In approximately 10% of fibrinogen molecules, one γ chain, termed γ′, γB, or γ^{57.5} has a twenty amino acid sequence⁷ substituted for the carboxyl terminal four amino acids found in the more common γ chain, sometimes termed γA or γ⁵⁰. This extension results from alternative mRNA processing,^{8,9} and disrupts the binding site for platelet integrin.^{10,11,12} Furthermore, the γ′ extension is highly anionic, containing sulfotyrosine residues¹³ and seven Asp and Glu residues, and mediates binding of γA/γ′ fibrinogen (also known as “peak 2” fibrinogen)¹⁴ to zymogen coagulation factor XIII^{15,16} and thrombin.¹⁷

Previous studies have shown that γA/γ′ fibrinogen forms clots that are more extensively crosslinked by factor XIIIa, a plasma transglutaminase, and are therefore resistant to breakdown by fibrinolytic enzymes, including tissue-type plasminogen activator.¹⁸ In addition, the binding of thrombin to γA/γ′ fibrin may provide an additional source of clot-bound thrombin.¹⁷ Clot-bound thrombin is active even in the presence of heparin, since clot-bound thrombin is resistant to heparin-catalyzed inhibition by antithrombin III.^{19,20}

Drouet et al.²³ hypothesized that the ratio of γA/γ′ fibrinogen to total fibrinogen may be a potential marker for cardiovascular risk. It has now been surprisingly discovered, however, that this ratio is less predictive of cardiovascular risk than the total level of γA/γ′ fibrinogen. Thus, the level of γA/γ′ fibrinogen has been found to constitute an independent risk factor for coronary artery disease.

It is therefore a primary objective of the present invention to provide a new means of detecting individuals at risk for coronary artery disease.

It is a further objective of the present invention to provide a means of detecting individuals at risk for coronary artery disease using a monoclonal antibody that is specific to γA/γ′ fibrinogen.

It is still a further objective of the present invention to provide an assay for γA/γ′ as a diagnostic test for coronary artery disease.

It is yet a further objective of the present invention to provide a hybridoma that produces antibody molecules that specifically immunoreact with a ligand-induced binding site on γA/γ′ fibrinogen.

It is still a further objective of the present invention to provide a diagnostic kit for detecting individuals at risk for coronary artery disease.

These and other objectives will become clear from the foregoing detailed description.

### SUMMARY OF THE INVENTION

The present invention is directed to a method and means for diagnosing and detecting coronary artery disease. It has now been found that elevated plasma levels of γA/γ′ fibrinogen are an independent risk factor for coronary artery disease. Based on this finding, diagnostic tests can be developed to detect individuals who are at risk for developing the disease.

The present invention further contemplates a method and diagnostic kit for the detection of γA/γ′ fibrinogen levels in patients. The kit preferably contains an antibody molecule or fragment thereof that is capable of specifically immunoreacting with a binding site in γAγ′ fibrinogen. In a preferred embodiment, the antibody composition contains a monoclonal antibody or fragment thereof that specifically immunoreacts with a binding site in γA/γ′ fibrinogen. Most preferably, the monoclonal antibody is capable of immunoreacting with the twenty carboxyl terminal amino acids of the γ′ chain of γA/γ′ fibrinogen.

A preferred method of the invention involves an ELISA assay using the monoclonal antibody immunoreactive with the γA/γ′ fibrinogen. An elevated level of γA/γ′ fibrinogen in the plasma would then be indicative of an increased risk of coronary artery disease.

### BRIEF DESCRIPTION OF THE DRAWINGS

FIG. 1 is a graph illustrating the distribution of γA/γ′ Fibrinogen Levels in Cases and Controls by Age. The mean levels of γA/γ′ fibrinogen±SD for each age group are shown for cases (black bars) and controls (stippled bars).

FIG. 2 is a graph illustrating the distribution of γA/γ′ Fibrinogen Levels in Cases and Controls by Quintile. The cases (black bars) and controls (stippled bars) were stratified in quintiles according to the γA/γ′ fibrinogen level.

### DETAILED DESCRIPTION OF THE INVENTION

Published data has shown that an elevated level of plasma fibrinogen is associated with an increased risk of coronary artery disease. It has also been hypothesized that the ratio of γA/γ′ to total fibrinogen may be a potential marker for cardiovascular risk. However, the present inventors have now discovered that the level of γA/γ′ fibrinogen in plasma constitutes an independent risk factor for coronary artery disease. The inventors have also found that the ratio of γA/γ′ fibrinogen to total fibrinogen is less predictive of cardiovascular risk than either the level of γA/γ′ fibrinogen or total fibrinogen. These findings can be used in constructing methods and means of diagnosing coronary artery disease.

The inventors have determined that compared to patients having γA/γ′ fibrinogen levels of <0.23 mg/ml, the risk of coronary artery disease increases significantly in patients with γA/γ′ fibrinogen levels>0.29 mg/ml, with a remarkable increase amongst patients with γA/γ′ fibrinogen levels of >0.41 mg/ml. These findings confirm the presence of a dose-response relation between the γA/γ′ fibrinogen level and the risk of coronary artery disease. This coronary artery disease risk factor is independent of total fibrinogen levels.

As used herein, the term “amino acid” relates to amino acid residues in the natural L-configuration. It should be noted that all amino acid residue sequences are represented herein by formulae whose left to right orientation is in the conventional direction of amino-terminus to carboxy-terminus. Furthermore, it should be noted that a dash at the beginning or end of an amino acid residue sequence indicates a bond to a further sequence of one or more amino acid residues up to a total of about fifty residues in the polypeptide chain.

As used herein, the terms “polypeptide” and “peptide” are used interchangeably to designate a linear series of no more than about 50 amino acid residues connected one to the other by peptide bonds between the alpha-amino and carboxy groups of adjacent residues.

As used herein, the term “protein” is used to designate a linear series of greater than 50 amino acid residues connected one to the other as in a polypeptide.

As used herein, the term “receptor” and “receptor protein” are used herein to indicate a biologically active proteinaceous molecule that specifically binds to (or with) other molecules.

As used herein, the term “ligand” refers to a molecule that contains a structural portion that is bound by specific interaction with a particular receptor protein.

As used herein, the term “antibody” refers to immunoglobulin molecules and immunologically active portions of immunoglobulin molecules, i.e., molecules that contain an antibody combining site or paratope.

As used herein, the phrase “antibody combining site” refers to the structural portion of an antibody molecule comprised of a heavy and light chain variable and hypervariable region(s) that specifically binds (immunoreacts with) antigen.

As used here, the terms “monoclonal antibody” or “monoclonal antibody composition” refer to an antibody molecule that contains only one species of antibody combining site capable of immunoreacting with a particular antigen. A monoclonal antibody composition thus typically displays a single binding affinity for any antigen with which it immunoreacts. A monoclonal antibody composition is typically composed of antibodies produced by clones of a single cell called a hybridoma that secretes (produces) only one type of antibody molecule. The hybridoma cell is formed by fusing an antibody-producing cell and a myeloma or other self-perpetuating cell line. Such antibodies were first described by Kohler and Milstein, Nature 256:495-497 (1975), the disclosure of which is herein incorporated by reference. An exemplary hybridoma technology is described by Niman et al., Proc. Natl. Acad. Sci. U.S.A., 80:4949-4953 (1983). Other methods of producing a monoclonal antibody, a hybridoma cell, or a hybridoma cell culture are also well known. See e.g., Antibodies: A Laboratory Manual, Harlow et al., Cold Spring Harbor Laboratory, 1988; or the method of isolating monoclonal antibodies from an immunological repertoise as described by Sasatry, et al, Proc. Natl. Acad. Sci. USA, 86:5728-5732 (1989); and Huse et al., Science, 246:1275-1281 (1981). The references cited are hereby incorporated herein by reference.

The hybridoma so prepared produces a supernate that can be screened for the presence of antibody molecules that immunoreact with γA/γ′ fibrinogen. Briefly, to form the hybridoma from which the monoclonal antibody composition is produced, a myeloma or other self-perpetuating cell line is fused with lymphocytes obtained from the spleen of a mammal hyperimmunized with an immunogen.

Immunogens are prepared by attaching the individual antigenic peptides or individual epitope peptides, or any peptide containing said epitope onto an immunogenic carrier molecule capable of inducing antibody synthesis in animals. An immunogen is defined herein as a substance of sufficient size that when introduced into an animal stimulate the production of antibodies reactive with the specific antigen or epitope. Immunogenic carrier is defined herein as a protein or other high molecular weight compound to which an antigen or epitope is conjugated in vitro and which renders the antigen or epitope capable of stimulating or increasing an immune response. Peptides containing the specific amino acid epitope are herein referred to as antigenic peptides.

The peptide antigens of the present invention may be synthesized using any suitable peptide synthesis technique, such as solid phase peptide synthesis chemistry following the method of Merrifield, J. Am. Chem. Soc. 85: 2149-2154. The peptides terminating in a carboxyl group are synthesized on the standard Merrifield resin. The peptides terminating in an amide group are synthesized on a 4-methyl benzhydrylamine resin. Hydrofluoric acid cleavage of the C-terminal amino acid residue from this resin yields a peptide containing an amide terminus. Such procedures are well known to persons skilled in the art. Linking amino acids such as cysteine are added, if necessary, to either the amino or carboxyl terminus of antigen peptides either during synthesis or chemically after synthesis to provide a free sulfhydryl group to facilitate coupling to an immunogenic carrier. The linking amino acid is added at the opposite end of the peptide sequence from the cleavage site to allow attachment to the carrier protein leaving the proteolytic cleavage site exposed. Internal molecular markers such as norleucine may also be added during the synthesis of antigenic peptides to evaluate the number of peptide molecules bound to the carrier. Norleucine is a preferred marker since it is not a usual amino acid component of natural proteins. The synthesized peptides are purified by preparative reverse phase high performance liquid chromatography (HPLC) with identity and purity being established by fast atom bombardment (FAB) mass spectrometry and amino acid analysis, techniques well known in the art.

The antigenic peptides are covalently coupled to high molecular weight carrier proteins which include, but are not limited to, bovine serum albumin (BSA), bovine thyroglobulin (BT), keyhole limpet hemocyanin (KLH), ovalbumin (OA), and the like, with BSA and BT being preferred. The antigenic peptides are coupled to the linking amino acid, cysteine, by maleimido-NHS-ester heterobifunctional coupling reagents which include, but are not limited to m-Maleimidobenzoyl-N-hydroxysuccinimide ester (MSB), m-Maleimidobenzoyl-sulfosuccinimide ester (Sulfo-MBS), Succinimidyl 4-(Maleimidomethyl)-cyclohexane-1-carboxylate (SMCC) sulfosuccinimidyl 4-(N-Maleimidomethyl)cyclohexane-1-carboxylate (Sulfo-SMCC), Succinimidiyl 4-(p-Maleimidophenyl) butryate, (SMPB), Sulfoccinimidiyl 4-(p-Maleimidophenyl) butyrate, (Sulfo-SMPB), with MBS and Sulfo-MBS being preferred.

It is preferred that the myeloma cell line used to prepare a hybridoma be from the same species as the lymphocytes. Typically, a mouse of the strain 129 GIX⁺ is a preferred mammal. Suitable mouse myelomas for use in the present invention include the hypoxanthine-aminopterin-thymidine-sensitive (HAT) cell lines P3X63-Ag8.653, and Sp2/0-Ag14 that are available from the American Type Culture Collection, Rockville, Md., under the designations CRL 1580 and CRL 1581, respectively.

Splenocytes are typically fused with myeloma cells using polyethylene glycol (PEG) 1500. Fused hybrids are selected by their sensitivity to HAT. Hybridomas producing a monoclonal antibody of this invention are identified using the enzyme linked immunosorbent assay (ELISA).

A monoclonal antibody of the present invention can also be produced by initiating a monoclonal hybridoma culture comprising a nutrient medium containing a hybridoma that produces and secretes antibody molecules of the appropriate polypeptide specificity. The culture is maintained under conditions and for a time period sufficient for the hybridoma to secrete the antibody molecules into the medium. The antibody-containing medium is then collected. The antibody-containing medium can then be further isolated by well known techniques.

Media useful for the preparation of these compositions are both well known in the art and commercially available and include synthetic culture media, inbred mice and the like. An exemplary synthetic medium is Dulbecco's minimal essential medium (DMEM; Dulbecco et al., Virol. 8:396 (1959)) supplemented with 4.5 gm/1 glucose, 20 mm glutamine, and 20% fetal calf serum. An exemplary inbred mouse strain is the BALB/c.

The monoclonal antibodies of this invention can be used in the therapeutic, diagnostic or in vitro methods disclosed herein where binding of γA/γ′ fibrinogen or a portion thereof is desired.

As noted above, the present invention encompasses methods and means of diagnosing individuals with elevated plasma levels of γA/γ′ fibrinogen as an independent risk factor for coronary artery disease. One embodiment of this invention contemplates a method of forming a monoclonal antibody that immunoreacts with a binding site on the protein. This method generally comprises the following steps:

(1) Immunizing an animal with the protein. This is typically accomplished by administering an immunologically effective amount i.e., an amount sufficient to produce an immune response, of immunogen to an immunologically competent mammal. Preferably, the mammal is a rodent such as a rabbit, rat or mouse. The mammal is then maintained for a time period sufficient for the mammal to produce cells secreting antibody molecules that immunoreact with the protein.

(2) Removing antibody-producing cells from the immunized animal and preparing a suspension of the cells. This is typically accomplished by removing the spleen of the mammal and mechanically separating the individual spleen cells in a physiologically tolerable medium using methods well known in the art.

(3) Treating the cells with a transforming agent to produce transformed antibody-producing cells. Transforming agents and their use to produce immortalized cell lines are well known in the art and include DNA viruses such as Epstein Barr Virus (EBV), Simian Virus 40 (SV40), Polyoma Virus and the like, RNA viruses such as Moloney Murine Leukemia Virus (Mo-MuLV), Rous Sarcoma Virus and the like, myeloma cells such as P3x63-Ag8.653, Sp2/O-Ag14 and the like.

(4) Cloning the transformed cells, preferably to monoclonality. The cloning is preferably performed in a tissue culture medium that will not sustain (support) non-transformed cells. When the transformed cells are hybridomas, this is typically performed by diluting and culturing in separate containers the mixture of unfused spleen cells, unfused myeloma cells, and fused cells (hybridomas) in a selective medium which will not support (sustain) the unfused myeloma cells for a time sufficient to allow death of the unfused cells (about one week).

(5) Evaluating the tissue culture medium of the cloned transformants for the presence of secreted antibody molecules that immunoreact with the cell surface receptor or the ligand when either is in non-bound form using well known immunological techniques.

(6) Selecting and growing in a tissue culture medium of the cloned transformants for the presence of secreted antibody molecules that immunoreact with γA/γ′ fibrinogen but do not immunoreact with γA/γA fibrinogen. This is followed by recovery of the desired antibody from the culture supernatant.

The monoclonal antibody compositions produced by the above method can be used, for example, in diagnostic and therapeutic modalities wherein formation of a γA/γ′ fibrinogen-containing immunoreaction product is desired. Hybridomas of the present invention are those which are characterized as having the capacity to produce an anti-γA/γ′ fibrinogen monoclonal antibody composition or a composition containing monoclonal antibody specific to portions of γA/γ′ fibrinogen. Methods for producing hybridomas producing (secreting) antibody molecules having a desired immunospecificity, i.e., having the ability to immunoreact with a particular protein, an identifiable epitope on a particular protein and/or a polypeptide, are well known in the art and are described further herein. Particularly applicable is the hybridoma technology described by Niman et al., Proc. Natl. Acad. Sci. USA, 80:4949-4953 (1983), and by Galfre et al., Meth. Enzymol., 73:3-46 (1981), the descriptions of which are herein incorporated by reference.

Any monoclonal antibody is appropriate for use in this invention so long as it is capable of immunoreacting with γA/γ′ fibrinogen or portions thereof, but not γA/γA fibrinogen. The methods for synthesizing such monoclonal antibodies are well known in the art. A particularly preferred anti-γA/γ′ fibrinogen monoclonal antibody of the invention is specifically directed against the γ′ chain of γA/γ′ fibrinogen. A most preferred monoclonal antibody is a synthetic peptide corresponding to the carboxyl terminal twenty amino acids of the γ′ chain of γA/γ′ fibrinogen: VRPEHPAETEYDSLYPEDDL (SEQ ID NO:1). This monoclonal antibody is herein designated as 2.G2.H9. 2.G2.H9 is preferably coupled to keyhole limpet hemocyanin as a carrier protein. However, other carrier proteins are also suitable for use in this invention. 2.G2.H9 recognizes γA/γ′ fibrinogen exclusively, and does not cross-react measurably with γA/γA fibrinogen.

The invention also contemplates the preparation of a diagnostic system in kit form in an amount sufficient for at least one assay, composition containing antibody or monoclonal antibody molecules or fragments thereof of the present invention, as a separately packaged reagent, together with a label that indicates the presence of an immunoreaction product.

In one embodiment, a diagnostic system is contemplated for assaying for the presence or a receptor-ligand complex, in a complex-containing vascular fluid sample, such as blood or plasma. The diagnostic system comprises a package containing antibody molecules that immunoreact with γA/γ′ fibrinogen but do not immunoreact with γA/γA fibrinogen.

For both an in vitro and in vivo embodiment, a diagnostic system of the present invention may include a label or indicating means capable of signaling the formation of a specifically bound complex containing an antibody molecule of the present invention. As used herein, the terms “label” and “indicating means” in their various grammatical forms refer to single atoms and molecules that are either directly or indirectly involved in the production of a detectable signal to indicate the presence of a complex. Any label or indicating means can be linked to or incorporated in an antibody molecule that is part of an antibody or monoclonal antibody composition of the present invention, or used separately, and those atoms or molecules can be used alone or in conjunction with additional reagents. Such labels are well-known in clinical diagnostic chemistry.

The linking of labels, i.e., labeling of, polypeptides and proteins is well known in the art. For instance, antibody molecules produced by a hybridoma can be labeled by metabolic incorporation of radioisotope-containing amino acids provided as a component in the culture medium. See, for example, Galfre et al., Meth. Enzymol., 73:3-46 (1981). The techniques of protein conjugation or coupling through activated functional groups are particularly applicable. See, for example, Auramease, et al., Scand. J. Immunol., Vol. 8, Suppl. 7:7-23 (1978), Rodwell et al., Biotech, 3:889-894 (1984), and U.S. Pat. No. 4,493,795.

The diagnostic systems can also include, preferably as a separate package, a specific binding agent. A “specific binding agent” is a molecular entity capable of selectively binding a reagent species of the present invention but is not itself an antibody molecule of the present invention. Exemplary specific binding agents are antibody molecules, complement proteins or fragments thereof, and the like. Preferably, the specific binding agent can bind the antibody molecule of this invention when it is present as part of a complex.

In preferred embodiments, the specific binding agent is labeled. However, when the diagnostic system includes a specific binding agent that is not labeled, the agent is typically used as an amplifying means or reagent. In these embodiments, the labeled specific binding agent is capable of specifically binding the amplifying means when the amplifying means is bound to a reagent species-containing complex.

The diagnostic kits of the present invention can be used in an “ELISA” format to detect, for example, the presence or quantity of γA/γ′ fibrinogen in a body fluid sample such as serum, plasma, or urine. “ELISA” refers to an enzyme-linked immunoabsorbent assay that employs an antibody or antigen bound to a solid phase and an enzyme-antigen or enzyme-antibody conjugate to detect and quantify the amount of an antigen or antibody present in a sample. A description of the ELISA technique is found in Chapter 22 of the 4^{th} Edition of Basic and Clinical Immunolog by D. P. Sites et al., published by Lange Medical Publications of Los Altos, Calif. In 1982 and in U.S. Pat. Nos. 3,654,090; 3,850,752; and 4,016,043, the disclosures of which are herein incorporated by reference.

Thus, in preferred embodiments of this invention, the antibody or antigen reagent component can be affixed to a solid matrix to form a solid support that is separately packaged in the subject diagnostic systems. The reagent is typically affixed to the solid matrix by adsorption from an aqueous medium, although other modes of affixation, well known to those skilled in the art, can be used. Useful solid matrices are well known in the art. Such materials include the cross-linked dextran available under the trademark SEPHADEX from Pharmacia Fine Chemicals (Piscataway, N.J.); agarose; polystyrene beads about 1 micron to about 5 millimeters in diameter available from Abbott Laboratories of North Chicago, Ill.; polyvinyl chloride, polystyrene, cross-linked polyacrylamide, nitrocellulose- or nylon-based webs such as sheets, strips or paddles; or tubes, plates or the wells of a microtiter plate such as those made from polystyrene or polyvinylchloride.

The reagent species, labeled specific binding agent or amplifying reagent of any diagnostic system described herein can be provided in solution, as a liquid dispersion or as a substantially dry powder, e.g., in lyophilized form. Where the indicating means is an enzyme, the enzyme's substrate can also be provided in a separate package of a system. A solid support such as the before-described microtiter plate and one or more buffers can also be included as separately packaged elements in this diagnostic assay system.

The packages discussed herein in relation to diagnostic systems are those customarily utilized in diagnostic systems. Such packages include glass and plastic (e.g., polyethylene, polypropylene and polycarbonate) bottles, vials, plastic and plastic-foil laminated envelopes and the like.

The present invention also contemplates any method that results in detecting γA/γ′ fibrinogen. The method for detecting γA/γ′ fibrinogen comprises the formation of an immunoreaction product between γA/γ′ fibrinogen and an anti-γA/γ′ fibrinogen antibody molecule, as disclosed herein, and the subsequent detection of the immunoreaction product so formed. The γA/γ′ fibrinogen to be detected can be present in a biological or vascular fluid sample, such as a blood sample, or can be present in a body tissue. Those skilled in the art will understand that there are numerous well known clinical diagnostic chemistry procedures that can be utilized to form detectable immunocomplexes. Thus, while exemplary assay methods are described herein, the invention is not so limited.

A method is contemplated for detecting in vivo in a human subject the presence of γA/γ′ fibrinogen. An effective amount of an antibody composition or a monoclonal antibody composition of the present invention containing anti-γA/γ′ fibrinogen molecules, linked to an in vivo detecting means, is intravenously administered into the subject in the form of a physiologically tolerable preparation. An effective amount of an antibody composition for in vivo detection of γA/γ′ fibrinogen is an amount sufficient to deliver and produce a blood concentration of anti-γA/γ′ fibrinogen antibody molecules of about 0.1 to about 10 mM.

The subject is then maintained for a predetermined time period sufficient for the labeled antibody molecules to react with the γA/γ′ fibrinogen and form a complex, and preferably for an additional time period sufficient for a substantial amount of any non-reacted antibody molecules to clear the body. The subject is then assayed for the presence and preferably location of any labeled complex that formed.

Various heterogeneous and homogeneous assay protocols can be employed, either competitive or non-competitive for detecting the presence and preferably amount of γA/γ′ fibrinogen in a body sample, preferably a body fluid sample, more preferably a vascular fluid sample such as blood. The method involves the admixture of a blood sample with antibody molecules that immunoreact with γA/γ′ fibrinogen but not with unbound molecules.

Biological assay conditions are those that maintain the biological activity of the antibody molecules and polypeptide molecules of this invention and the γA/γ′ fibrinogen sought to be assayed. Those conditions include a temperature range of about 4-45° C. at a pH value of 5-9, and an ionic strength varying from that of distilled water to that of about one molar sodium chloride, preferably about that of physiological saline.

The following example is offered to illustrate but not limit the invention. Thus, it is presented with the understanding that various formulation modifications as well as method of delivery modifications may be made and still are within the spirit of the invention.

### EXAMPLE 1

### Association Between γA/γ′ Fibrinogen Levels and Coronary Artery Disease

Purpose

The purpose of this case-control study was to determine whether there is an association between γA/γ′ fibrinogen levels and coronary artery disease. The study also examined the dependence on this association on total fibrinogen levels.

Methods

Patients and Controls

Patients who were referred for elective, outpatient diagnostic cardiac catheterizations were considered candidates for the study. Indications for catheterization included anginal chest pain, positive stress test, valvular heart disease, and preoperative clearance prior to non-cardiac surgery in patients suspected of ischemic heart disease. Patients were entered sequentially during two different sampling intervals; April through August, 1996 and October through December, 1997. All patients signed an informed consent form approved by the Pennsylvania State University College of Medicine Institutional Review Board. Control blood samples were obtained from anonymous blood donors between the ages of 41 and 80 at the Pennsylvania State University College of Medicine blood bank, and were matched to the cases by age and gender.

All cardiac catheterizations were performed via the femoral approach using the Judkin's technique, with arterial blood samples obtained immediately after arterial access before the administration of heparin. Coronary cineangiography was performed in standard projections. All studies were interpreted qualitatively by two angiographers, with a third angiographer reviewing films as required. All discrepancies in interpretation were settled by consensus. A patient was considered to have significant coronary artery disease if she/he had a luminal narrowing lesion of 50% or greater in at least one major coronary artery or branch.

Laboratory Studies

Blood was collected in standard citrate anticoagulant (10%). Plasma was prepared by centrifugation at 1000×g for 10 minutes at room temperature and was stored at −70° C. Samples were thawed only once for use in the assay, due to the propensity of fibrinogen to form a cold-insoluble precipitate upon freeze-thaw.

A monoclonal antibody directed against the γ′ chain (2.G2.H9) was developed at the Pennsylvania State University Biotechnology Institute by Dr. William Scheuchenzuber. The immunogen was a synthetic peptide corresponding to the carboxyl terminal twenty amino acids of the γ′ chain. VRPEHPAETEYDSLYPEDDL (SEQ ID NO:1), coupled to keyhole limpet hemocyanin as a carrier protein. 2.G2.H9 recognizes γA/γ′ fibrinogen exclusively, and does not cross-react measurably with γA/γA fibrinogen (data not shown). 2.G2.H9 was purified from 30 ml of a concentrated bioreactor preparation by precipitation with 50% ammonium sulfate followed by DEAE-cellulose chromatography in 0.01 M potassium phosphate, pH 8.0.

Monoclonal antibody 2.G2.H9 was used to develop an ELISA assay specific for γA/γ′ fibrinogen, using a buffer and blocking system described previously for the measurement of annexin V in plasma.²¹ 2.G2.H9 (1.5 μg/ml) was used as the capture antibody, and bound γA/γ′ fibrinogen was detected with a commercial rabbit antihuman fibrinogen immunoglobulin fraction (Accurate Chemical & Scientific Corp., Westbury, N.Y.) coupled to biotin. The ELISA was developed with a streptavidin-alkaline phosphatase conjugate (Life Technologies, Gaithersburg, Md.) incubated with a phosphatase substrate (Sigma Chemical Co., St. Louis, Mo.).

γA/γ′ fibrinogen was purified as described previously using DEAE-cellulose chromatography.^{14 18} Pooled human plasma was heat-defibrinated for 30 minutes at 56° C. and centrifuged at 100,000×g for 30 minutes at 4° C. ELISA standards were prepared by reconstituting defibrinated plasma with purified γA/γ′ fibrinogen. Plasma samples and standards were diluted 1:1000 for the assay.

Total fibrinogen was assayed similarly, except that rabbit anti-human fibrinogen was used as capture antibody, heat-defibrinated plasma was reconstituted with unfractionated fibrinogen rather than γA/γ′ fibrinogen, and samples were diluted 1:10,000 for the assay.

Statistical Analysis

Statistical analysis was performed using SPSS (Statistical Package for Social Sciences, Inc., Chicago, Ill.) for Windows software. Descriptive statistics (average and proportion) were used to assess the distributional characteristics of the patients. The Student t-test and Chi-square test were used to compare two means and proportions. The Pearson correlation coefficient was used to assess the correlation between two continuous variables. A two-sided probability (P) value≦0.05 was required for statistical significance.

Multiple logistic regression analysis was used to examine the associate between coronary artery disease and γA/γ′ fibrinogen levels, while adjusting for confounding factors such as age and gender. The independent variables, the variables of interest, were the γA/γ′ fibrinogen level, the total fibrinogen level, or the ratio of the two. The fibrinogen variables were entered into the model as a categorical measure based upon on quintiles that were not subject to investigator bias. In the risk analysis, the group with the lowest fibrinogen levels was compared to each of the higher quintiles to calculate the odds ratios. Two logistic regression models were fitted. Model 1 tested the association of coronary artery disease with γA/γ′ fibrinogen alone while controlling for the effect of age and gender. Model 2 tested whether this association was independent of total fibrinogen levels while controlling for the effect of age and gender. Additionally, a stepwise approach was used to assess whether the ratio of γA/γ′ fibrinogen to total fibrinogen or γA/γ′ fibrinogen alone is a stronger predictor for the risk of coronary artery disease, while controlling for age, gender, and total fibrinogen levels. Odds ratios (OR) were calculated to measure the magnitude of this association. Ninety-five percent confidence intervals (CI) for the adjusted odds ratios were based on the Cornfield method.²² The goodness-of-fit of the model was tested by the likelihood ratio test.

Results

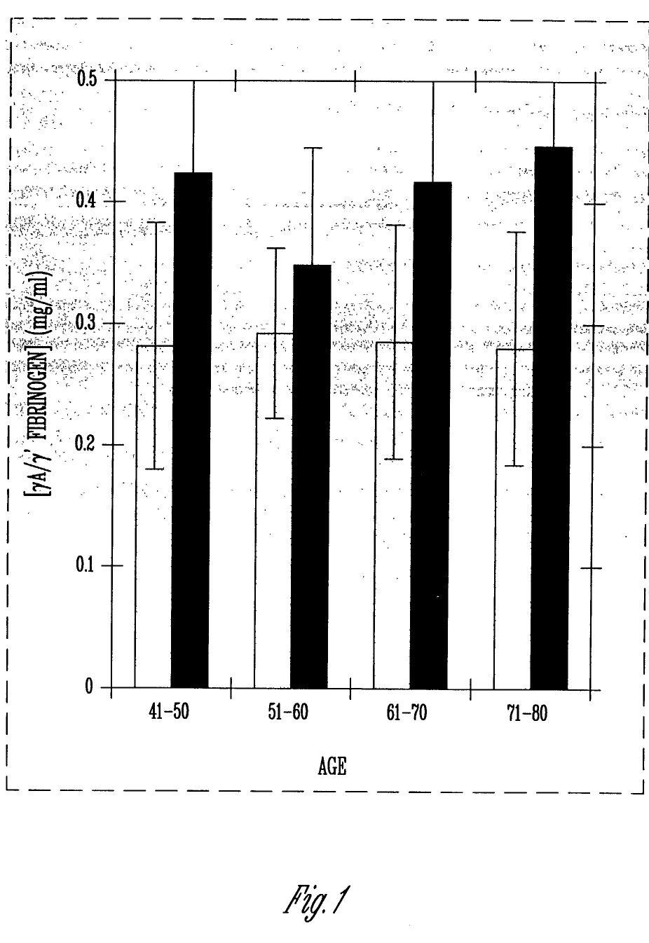

Table 1 presents the age and gender distribution of the case and control groups. The mean age (year) was slightly higher in cases than in controls (62.8 vs. 60.0 years, P=0.04). Overall, only one-third of the study subjects were female, and a slightly higher proportion of females was found in controls. The mean (±SD) γA/γ′ fibrinogen level (mg/ml) in the cases was 0.414±0.149, as compared to 0.286±0.088 in the controls (P<0.001), an increase of 1.45-fold. In contrast, total fibrinogen levels were elevated only 1.22-fold in cases compared to controls. Female cases and controls both had higher levels of total fibrinogen and γA/γ′ fibrinogen compared to their male counterparts, but this gender difference was not seen in the ratio of γA/γ′ fibrinogen to total fibrinogen (Table 2). γA/γ′ fibrinogen levels did not show a significant trend either upward or downward with age (P=0.12) (FIG. 1), and were remarkably stable among the controls at all age intervals tested (P=0.99). There was a weak but significant correlation between γA/γ′ fibrinogen and total fibrinogen levels (r=0.16, P=0.02). Mean γA/γ′ fibrinogen levels were consistently elevated in cases compared to controls across all age and gender groups.

TABLE 1
Gender and age distribution of case and control groups.

| | Cases (N = 91) | Controls (N = 120) | |
|---|---|---|---|
| Variable | n (%) | n (%) | P value |
| Gender | | | |
| Female | 30 (33.0) | 46 (38.3) | 0.26 |
| Male | 61 (67.0) | 74 (61.7) | |
| Age | | | |
| 41-50 | 12 (13.2) | 24 (20.0) | 0.37 |
| 51-60 | 23 (25.3) | 34 (28.3) | |
| 61-70 | 32 (35.2) | 40 (33.3) | |
| 71-80 | 24 (26.4) | 22 (18.3) | |

TABLE 2
Total and γA/γ′ fibrinogen levels in cases and controls by gender.

| | Mean ± SD | | |
|---|---|---|---|
| Variable | Cases (N = 91) | Controls (N = 120) | P Value |
| Total fibrinogen, mg/ml | | | |
| Female | 5.20 ± 1.28 | 4.11 ± 0.89 | <0.001 |
| Male | 4.64 ± 1.41 | 3.86 ± 1.31 | 0.001 |
| γA/γ′ fibrinogen, mg/ml | | | |
| Female | 0.46 ± 0.15 | 0.30 ± 0.10 | <0.001 |
| Male | 0.39 ± 0.14 | 0.28 ± 0.08 | <0.001 |
| Ratio | | | |
| Female | 9.51 ± 4.17 | 7.32 ± 2.41 | 0.005 |
| Male | 9.65 ± 5.75 | 7.89 ± 3.25 | 0.027 |

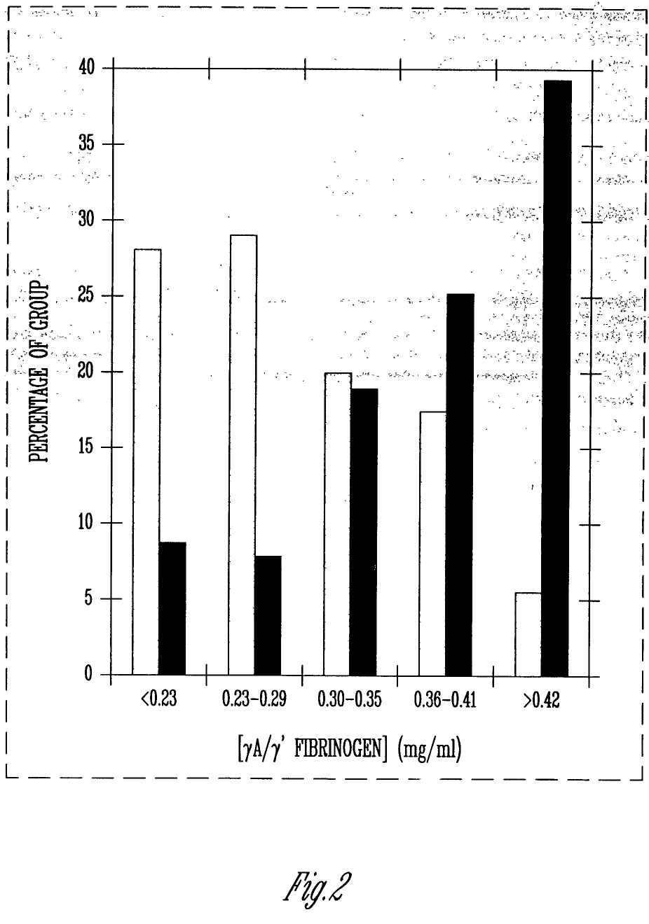

When γA/γ′ fibrinogen levels were ranked by quintile, a distinct difference in distribution was found between cases and controls (FIG. 2). The cases showed a distribution of γA/γ′ fibrinogen skewed towards higher concentrations, such that in the highest concentration quintile, 36 cases were represented compared to only 7 controls. The odds ratio for individuals who had γA/γ′ fibrinogen levels in the highest quintile, as compared to those who had γA/γ′ fibrinogen levels in the lowest quintile, was 21.9 (95% CI=7.1-66.8) before adjusting for any confounding factors, a remarkably high odds ratio for a single variable.

Table 3 presents the two multiple logistic regression models examining the association between coronary artery disease and γA/γ′ fibrinogen levels. The lowest quintile group was used as a reference for comparison in both models. In model 1, the risk of coronary artery disease increased significantly with the increased quintile of the γA/γ′ fibrinogen levels, after controlling for age and gender. A large increase was noted among subjects with γA/γ′ fibrinogen levels in the highest quintile (OR=27.0, 95% CI=7.7-94.1). When total fibrinogen was included in the model, the odds ratio for γA/γ′ fibrinogen levels declined slightly by 5%, but still remained statistically significant, indicating an independent effect of γA/γ′ fibrinogen levels from total fibrinogen. The stepwise analysis showed that the γA/γ′ fibrinogen is a stronger predictor than the ratio of γA/γ′ fibrinogen to total fibrinogen for coronary artery disease, after controlling for age, gender, and total fibrinogen. No interaction was found between any of the variables examined.

TABLE 3
Association of coronary artery disease with γA/γ′ fibrinogen: The
results of multiple logistic regression analysis.

| Variable | Odds Ratio | 95% CI |
|---|---|---|
| Model 1* | | |
| γA/γ′ fibrinogen quintile (mg/ml) | | |
| <0.23 | 1.0 | — |
| | (reference) | |
| 0.23-0.29 | 0.9 | 0.3-2.7 |
| 0.30-0.35 | 3.2 | 1.2-8.7 |
| 0.36-0.41 | 4.9 | 1.8-13.3 |
| >0.42 | 28.3 | 8.7-92.1 |
| Model 2* | | |
| Total fibrinogen quintile (mg/ml) | | |
| <3.11 | 1.0 | — |
| | (reference) | |
| 3.11-3.86 | 1.2 | 0.4-4.0 |
| 3.87-4.56 | 1.7 | 0.5-5.5 |
| 4.57-5.54 | 3.8 | 1.2-12.2 |
| >5.5 | 7.7 | 2.4-25.1 |
| γA/γ′ fibrinogen quintile (mg/ml) | | |
| <0.23 | 1.0 | — |
| | (reference) | |
| 0.23-0.29 | 0.6 | 0.2-2.2 |
| 0.30-0.35 | 2.2 | 0.8-6.6 |
| 0.36-0.41 | 3.0 | 1.0-8.9 |
| >0.42 | 27.0 | 7.7-94.1 |

*Controlling for age and gender.

Discussion

The data indicate that the level of γA/γ′ fibrinogen in plasma constitutes an independent risk factor for coronary artery disease. This conclusion differs significantly from that of Drouet et al.,²³ who hypothesized that the ratio of γA/γ′ fibrinogen to total fibrinogen may be a potential marker for cardiovascular risk. The present data indicate that the ratio of the two is less predictive of risk than either the level of γA/γ′ fibrinogen or total fibrinogen. In addition, the study of Drouet et al.²³ showed an unexplained bimodal distribution of γA/γ′ fibrinogen to total fibrinogen ratio that was not apparent in the present study. Furthermore, no risk assessment data was presented to substantiate their hypothesis that the ratio of γA/γ′ fibrinogen to total fibrinogen may be a marker of cardiovascular risk.

A limitation of the present study is that it was not possible to include an extensive number of potential confounding factors, since the control blood samples were obtained from anonymous blood donors who were identified only by age and gender. It is possible that other variables (such as smoking, exercise, hypertension, alcohol intake, and obesity) that influence total fibrinogen levels may also have an impact on γA/γ′ fibrinogen levels.

The factors that regulate the levels of γA/γ′ fibrinogen are, for the most part, uncharacterized. The levels of total fibrinogen have been correlated with promoter polymophisms;^{24 25} however, as shown in the present study, γA/γ′ fibrinogen levels appear to vary independently of total fibrinogen levels. This may be explained by the fact that synthesis of the β chained mRNA is the rate-limiting factor in total fibrinogen expression,²⁶ whereas expression of the γ′ chain mRNA is more likely to be affected by the cleavage of intron/exon boundaries and polyadenylation sites in the mRNA by spliceosomes and polyadenylation enzymes, respectively.^{8,9} The processing events that give rise to the γ′ mRNA are also liver-specific, since the γ′ mRNA is not found in other tissues that express γA mRNA.²⁷ Mechanistically, the relative levels of γA versus γ′ mRNA may be the result of direct competition between spliceosomes that remove the ninth introl encoding the carboxyl terminus of the γ′ chain versus enzymes that cleave and polyadenylate the 3′ end of the γ′ mRNA within the ninth introl.⁸ Presumably, elevated levels of spliceosomes would favor removal of the ninth introl, increasing the level of γA mRNA. In contrast, elevated levels of polyadenylation activity would favor termination of the mRNA within the ninth intron, increasing the level of γ′ mRNA. Therefore, an understanding of the regulation of γA/γ′ fibrinogen levels will likely come from future investigations of ninth intron boundary polymorphisms, polyadenylation recognition sites, and liver-specific mRNA spliceosome and polyadenylation enzymes rather than the fibrinogen gene promoters.

Based on the results of this study, it is apparent that γA/γ′ fibrinogen levels are highly predictive of the risk of coronary artery disease, and may be used as a tool in the risk assessment of coronary artery disease.

Having described the invention with reference to particular compositions, theories of effectiveness, and the like, it will be apparent to those of skill in the art that it is not intended that the invention be limited by such illustrative embodiments or mechanisms, and that modifications can be made without departing from the scope or spirit of the invention, as defined by the appended claims. It is intended that all such obvious modifications and variations be included within the scope of the present invention as defined in the appended claims. The claims are meant to cover the claimed components and steps in any sequence, which is effective to meet the objectives there intended, unless the context specifically indicates to the contrary.

All articles cited herein and in the following list are hereby expressly incorporated in their entirety by reference.

Citations

1. Wilhelmsen L., Svardsudd K., Korsan-Bengtsen K., Larsson B., Welin L., Tibblin G. Fibrinogen as a risk factor for stroke and myocardial infarction. N. Engl. J. Med. 1984; 311:501-5.

2. Maresca, G., Di Blasio A., Marchioli R., Di Minno G. Measuring plasma fibrinogen to predict stroke and myocardial infarction: an update.

Arterioscler Thromb. Vasc. Biol. 1999; 19:1368-77.

3. Grieninger G., Hertzberg K M., Pindyck J., Fibrinogen synthesis in serum-free hepatocyte cultures: stimulation by glucocorticoids. Proc. Natl. Acad. Sci. USA 1978; 75:5506-10.

4. Wolfenstein-Todel C., Mosesson M W. Human plasma fibrinogen heterogeneity; evidence for an extended carboxyl-terminal sequence in a normal γ chain variant (γ′). Proc. Natl. Acad. Sci. USA 1980; 77:5069-73.

5. Francis C W., Marder V J., Martin S E. Demonstration of a large molecular weight variant of the 7 chain of normal human plasma fibrinogen. J. Biol. Chem. 1980; 255:5599-604.

6. Francis C W., Kraus D H., Marder V J. Structural and chromatographic heterogeneity of normal plasma fibrinogen associated with the presence of three types with distinct molecular weights. Biochim. Biophys. Acta. 1983; 744:155-64.

7. Wolfenstein-Todel C., Mosesson M W. Carboxy-terminal amino acid sequence of a human fibrinogen γ-chain variant (γ′). Biochemistry 1981; 20:6146-9.

8. Chung D W., Davie E W. γ and γ chains of human fibrinogen are produced by alternative mRNA processing. Biochemistry 1984; 23:4232-6.

9. Fornace A J., Cummings D E., Comeau D M., Kant J A., Crabtree G R. Structure of the human γ-fibrinogen gene: alternate mRNA splicing near the 3′ end of the gene produces γA and γB forms of γ-fibrinogen. J. Biol. Chem 1984; 259:12826-30.

10. Farrell D H., Thiagarajan P., Chung, D W., Davie E W. Role of fibrinogen α and γ chain sites in platelet aggregation. Proc. Natl. Acad. Sci. USA 1992; 89:10729-32.

11. Lawrence S O., Wright T W., Francis C W., Fay P J., Haidaris P J. Purification and functional characterization of homodimeric γB-γB fibrinogen from rat plasma. Blood 1993; 82:2406-13.

12. Farrell D H., Thiagarajan P. Binding of recombinant fibrinogen mutants to platelets. J. Biol. Chem. 1994; 269:226-31.

13. Farrell D H., Mulvihill E R., Huang S., Chung D W., Davie E W. Recombinant human fibrinogen and sulfation of the γ′ chain. Biochemistry 1991; 30:9414-20.

14. Mosesson M W., Finlayson J S., Umfleet R A. Human fibrinogen heterogeneities: identification of γ chain variants. J. Biol. Chem. 1972; 247:5223-7.

15. Siebenlist K R., Meh D A., Mosesson M W. Plasma factor XIII binds specifically to fibrinogen molecules containing γ′ chains. Biochemistry 1996; 35:10448-53.

16. Moaddel M., Farrell D H., Daugherty M A., Fried M G. Interactions of human fibrinogens with factor XIII: roles of calcium and the γ′ peptide. Biochemistry 2000; 39:6698-705.

17. Meh D A., Siebenlist K R., Mosesson M W. Identification and characterization of thrombin binding sites on fibrin. J. Biol. Chem. 1996; 271:23121-5.

18. Falls L A., Farrell D H. Resistance of γA/γ′ fibrin clots to fibrinolysis. J. Biol. Chem. 1997; 272:14251-6.

19. Hogg P J., Jackson C M. Fibrin monomer protects thrombin from inactivation by heparin-antithrombin III: implications for heparin efficacy. Proc. Natl. Acad. Sci. USA 1989; 86:3619-23.

20. Weitz J I., Hudoba M., Massel D., Maraganore J., Hirsch J. Clot-bound thrombin is protected from inhibition by heparin-antithrombin III but is susceptible to inactivation by antithrombin III-independent inhibitors. J. Clin. Invest 1990; 86:385-91.

21. Flaherty M J., West S., Heimark R L., Fujikawa K., Tait J F. Placental anticoagulant protein-I: measurement in extracellular fluids and cells of the hemostatic system. J. Lab. Clin. Med. 1990; 115:174-81.

22. Cornfield J. Statistical problems arising from retrospective studies. In: Nryman J. ed. Proceedings of the Third Berkeley Symposium. Vol. 4. Berkeley: University of California Press. 1956; 133-48.

23. Drouet L., Paolucci F., Pasqualini N. Laprade M., Ripoll L., Mazoyer E., Bal dit Sollier C., Vanhove, N. Plasma γA/γ′ fibrinogen ratio, a marker of arterial thrombotic activity: a new potential cardiovascular risk factor? Blood Coag. Fibrinolysis 1999; 10:S35-9.

24. Thomas A E., Green F R., Kelleher C H., Wilkes H C., Brennan P J., Meade T W., Humphries S E. Variation in the promoter region of the beta fibrinogen gene is associated with plasma fibrinogen levels in smokers and non-smokers. Thromb. Haemost 1991; 65:487-90.

25. Fowkes F G., Connor J M., Smith F B., Wood J., Donnan P T., Lowe G D. Fibrinogen genotype and risk of peripheral atherosclerosis. Lancet 1992; 339:693-6.

26. Yu S., Sher B., Kudryk B., Redman C M. Fibrinogen precursors: order of assembly of fibrinogen chains. J. Biol. Chem. 1984; 259:10574-81.

27. Haidaris P J., Courtney M A. Tissue-specific and ubiquitous expression of fibrinogen γ chain mRNA. Blood Coag. Fibrinolysis 1990; 1:433-7.

For the above-stated reasons, it is submitted that the present invention accomplishes at least all of its stated objectives.

What is claimed is:

1. A method of detecting an individual at risk for coronary artery disease comprising: obtaining a biological sample from said individual; and assaying for the level of γA/γ′ fibrinogen present in said sample, wherein an elevated γA/γ′ fibrinogen level as compared with that of a person with no risk factor for coronary artery disease, is associated with an increased risk of developing coronary artery disease.

2. The method of claim 1 wherein the assaying includes the steps of: (a) contacting the biological sample with an antibody reactive only with γA/γ′ fibrinogen or parts thereof to form a complex; and (b) detecting said complex.

3. The method of claim 2 wherein the antibody is a monoclonal antibody.

4. The method of claim 3 wherein the monoclonal antibody is reactive with the carboxyl terminal twenty amino acids of the γ′ chain of γA/γ′ fibrinogen.

5. The method of claim 4 wherein the monoclonal antibody binds to SEQ ID NO:1.

6. The method of claim 3 wherein the monoclonal antibody is bound or captured to an antigen in said biological sample.

7. The method of claim 2 wherein the detecting step further includes the substep of linking or incorporating a label into the antibody.

8. The method of claim 7 wherein the label is a radioisotope-containing amino acid.

9. The method of claim 1 wherein the elevated γA/γ′ fibrinogen level is greater than 0.29 mg/ml.

10. The method of claim 1 wherein the elevated γA/γ′ fibrinogen level is greater than 0.41 mg/ml.

11. A monoclonal antibody which reacts with γA/γ′ fibrinogen or portions thereof.

12. The monoclonal antibody of claim 11 which reacts with the γ′ chain of γA/γ′ fibrinogen.

13. The monoclonal antibody of claim 11 that does not cross-react measurably with γA/γA fibrinogen.

14. The monoclonal antibody of claim 12 which binds the carboxyl terminal twenty amino acids of the γ′ chain of γA/γ′ fibrinogen.

15. The monoclonal antibody of claim 14 which binds SEQ ID NO:1.

16. A hybridoma that produces antibody molecules that specifically immunoreact with a binding site on γA/γ′ fibrinogen.

17. A method for detecting in vivo the presence of a γA/γ′ fibrinogen receptor comprising the steps of: (a) intravenously administering to an animal subject an effective amount of a monoclonal antibody composition comprising antibody molecules that immunoreact with γA/γ′ fibrinogen; (b) maintaining the administered subject for a predetermined time period sufficient for said antibody molecules to immunoreact with said γA/γ′ fibrinogen in vivo and form an immunoreaction product; and (c) assaying for the presence of any in vivo immunoreaction product formed in step (b) and thereby the presence of said γA/γ′ fibrinogen in said subject.

18. The method of claim 17 wherein the antibody molecules are administered in an amount sufficient to deliver and produce a blood concentration of antibody molecules of about 0.1-10 mM.

19. The method of claim 17 wherein the administered subject is maintained for a time sufficient for a substantial amount of any non-reacted antibody molecules to clear the body.

20. A kit for determining whether a biological sample contains γA/γ′ fibrinogen comprising: (a) a monoclonal antibody which reacts with γA/γ′ fibrinogen or portions thereof to form a complex; and (b) a label or other indicating means capable of signaling the formation of complex.

21. The kit of claim 20 wherein the monoclonal antibody reacts with the γ′ chain of γA/γ′ fibrinogen.

22. The kit of claim 20 wherein the monoclonal antibody binds the carboxyl terminal twenty amino acids of the γ′ chain of γA/γ′ fibrinogen.

23. The kit of claim 22 which binds SEQ ID NO:1.

24. The kit of claim 20 further including a specific binding agent.

25. The kit of claim 24 wherein the specific binding agent is selected from the group consisting of antibody molecules, complement proteins, and fragments thereof.

26. The kit of claim 20 wherein the specific binding agent is labeled.


SEQUENCE LISTING (1 sequence)

SEQ ID NO: 1 - protein, 20 aa, Artificial Sequence
  The sequence is completely synthesized.
VRPEHPAETEYDSLYPEDDL